(19) United States
(12) Patent Application Publication
(10) Pub. No.: US 2022/0296308 A1
(43) Pub. Date: Sep. 22, 2022

(54) DIRECTIONAL-DEVICE INTRABODY PLACEMENT SYSTEMS AND RELATED METHODS

(71) Applicant: ClearPoint Neuro, Inc., Irvine, CA (US)

(72) Inventors: Lyubomir Georgiev Zagorchev, Burlington, MA (US); Roberto Felipe Rubio, San Marcos, CA (US); Edwin Kelly, Atlanta, GA (US)

(21) Appl. No.: 17/590,153

(22) Filed: Feb. 1, 2022

Related U.S. Application Data
(60) Provisional application No. 63/161,650, filed on Mar. 16, 2021.

(51) Int. Cl. A61B 34/20; A61N 1/05; A61B 17/34; G06T 7/73; G16H 30/20; G16H 30/40

(52) U.S. Cl. CPC A61B 34/20; A61N 1/0534; A61B 17/3468; G06T 7/75; G16H 30/20; G16H 30/40; A61B 2034/2065; A61B 2090/374

(57) ABSTRACT

Methods and systems that identify an intrabody orientation of a directional medical device, such as a deep brain stimulation (DBS) lead, use a mesh model. The mesh model is a shape-constrained trained model of fused post-operative MRI and CT image data of brains from a set of patients defined by intensity of artifact patterns corresponding to a passive orientation marker on a physical device.

### RELATED APPLICATIONS

This patent application claims the benefit of and priority to U.S. Provisional Patent Application Ser. No. 63/161,650 filed Mar. 16, 2021, the contents of which are hereby incorporated by reference as if recited in full herein.

### FIELD OF THE INVENTION

The present invention relates to surgical image-guided systems.

BACKGROUND

Deep Brain Stimulation (DBS) requires accurate electrode implantation for optimal treatment outcomes. A direct relationship between electrode placement and clinical outcome has been suggested in several studies. See, Dembek et. al., Probabilistic mapping of deep brain stimulation effects in essential tremor, Neurolmage: Clinical, 13, pp. 164-173, 2017; Eisenstein et. al., Functional anatomy of subthalamic nucleus stimulation in Parkinson disease, Annals of neurology, 76(2), pp. 279-295, 2014; Garcia-Garcia et. al., Stimulation sites in the subthalamic nucleus and clinical improvement in Parkinson's disease: a new approach for active contact localization, Journal of neurosurgery, 125(5), pp. 1068-1079, 2016; Horn et. al., Connectivity predicts deep brain stimulation outcome in Parkinson disease, Annals of neurology, 82(1), pp. 67-78, 2017c; Mosley et. al., The site of stimulation moderates neuropsychiatric symptoms after subthalamic deep brain stimulation for Parkinson's disease, Neurolmage: Clinical, 18, pp. 996-1006, 2018b. The contents of these documents are hereby incorporated by reference as if recited in full herein.

Accurate electrode localization post-implantation is even more important in the case of directional DBS leads that allow for axial steering of applied neurostimulation. A recent manuscript suggests that the variability of directional lead implantation ranges from −90 to 90 degrees with respect to targeted orientation in a large number of patients. See, Dembek et. al., Directional DBS leads show large deviations from their intended implantation orientation. Parkinsonism & related disorders, 67, pp. 117-121, 2019; Methods that can detect lead orientation from post-operative CT have been developed. See, Hellerbach et. al., DiODe: directional orientation detection of segmented deep brain stimulation leads: a sequential algorithm based on CT imaging, Stereotactic and functional neurosurgery, 96(5), pp.335-341, 2018. The contents of these documents are hereby incorporated by reference as if recited in full herein.

MRI-guided DBS implantation can account for brain shift and provide superior accuracy for lead placement. However, existing implantation techniques do not provide support for lead orientation with respect to patient-specific neuroanatomy. Furthermore, manual lead orientation on the scalp surface does not necessarily translate to expected orientation at target depth as the lead can torque and deviate as it advances to target. See, e.g., Dembek, et. al., Directional DBS leads show large deviations from their intended implantation orientation. Parkinsonism & related disorders, 67, pp.117-121, 2019.

There remains a need for methods and systems for use with image guided systems that are capable of identifying and/or confirming orientation of directional devices, such as deep brain stimulation leads, before, during and/or after implantation to facilitate effective therapies.

### SUMMARY

Embodiments of the present invention are directed to systems, methods and computer program products for intrabody positioning of an orientation-specific directional medical device and/or verifying orientation of placement of directional medical devices, such as deep brain stimulation leads, using an image guided surgical system.

Embodiments of the present invention employ a shape-constrained mesh model of a directional medical device that can provide point to point correspondence of orientation in an intrabody MRI and/or CT image of a corresponding physical medical device.

Embodiments of the present invention are directed to an image-guided surgical system. The system includes a workstation comprising a display and a computer system in communication with or at least partially onboard the workstation. The computer system is configured to: provide a mesh model of a directional medical device; and determine an intrabody orientation of the directional medical device from CT and/or MRI image data.

The mesh model can be a shape-constrained trained model of fused post-operative MRI and CT image data of a set of patients having implanted respective directional medical devices using intensity of artifact patterns corresponding to a passive orientation marker on the respective directional medical device

The directional medical device can be a deep brain stimulation lead with a plurality of circumferentially spaced apart electrodes aligned in at least one longitudinal position of the directional medical device.

The system can include the directional medical device and a peel away sheath (PAS) coupled to the directional medical device. The PAS can have an elongate orientation marker that extends longitudinally over at least a major portion of an intrabody length of the directional medical device.

The computer system can be configured to determine the intrabody orientation directly from new MRI image data using the trained model without requiring post-operative, post-implantation CT images and without using any template.

The mesh model can be a high-resolution mesh model that is shape-constrained and can be deformable and defined based on geometry and topology of the directional medical device.

The mesh model can have a contiguous configuration of a plurality of triangular mesh elements. The intensity of artifact patterns can be intensity profiles perpendicular to the triangular mesh elements.

The computer system can be configured to generate output identifying a deviation from a desired orientation of the directional medical lead with respect to target tissue based on the determined orientation.

The computer system can be further configured to: provide a mesh brain model comprising vertices; select a subset of anatomical brain structures of interest relevant to a target intrabody site using the mesh brain model; evaluate quantitative indices derived from at least some of the mesh vertices; and define a patient-specific anatomy-based trajectory and/or a position and orientation of a directional medical device using the quantitative indices.

The quantitative indices can include one or more of a center of mass, a principal axis and a curvature of the mesh brain model and/or mesh elements thereof

The system can be used in combination with a CT or MRI scanner. The workstation can be in communication with the CT or MRI scanner. The workstation can have a DICOM interface that receives images from the CT or MRI scanner to provide the at least one image for the surgical system.

Embodiments of the present invention are directed to methods of identifying a deep brain stimulation lead orientation. The methods include: (electronically) providing a mesh brain model comprising vertices; (electronically) selecting a subset of anatomical brain structures of interest relevant to a target intrabody site using the mesh brain model; (electronically) evaluating quantitative indices derived from at least some of the mesh vertices; and (electronically) defining a patient-specific anatomy-based trajectory and/or a position and orientation of the DBS lead using the quantitative indices.

The quantitative indices can include one or more of: a center of mass, a principal axis and a curvature of the mesh brain model and/or mesh elements thereof

Yet other embodiments are directed to methods of identifying lead orientation of a deep brain stimulation (DBS) lead. The methods include providing a mesh model of the DBS lead. The mesh model is a shape-constrained trained model of fused post-operative MRI and CT image data of brains from a set of patients defined by intensity of artifact patterns corresponding to a passive orientation marker on respective actual corresponding to the DBS leads. The methods also include placing a DBS lead in a brain of a patient using an MRI image-guided surgical system; obtaining MRI image data of the brain of the patient; applying the mesh model to the MRI image data; and identifying orientation of the DBS lead in the brain of the patient using the applied mesh model.

The DBS lead can have a passive orientation marker longitudinally spaced apart from a plurality of circumferentially spaced apart electrodes. The circumferentially spaced apart electrodes can be aligned in at least one longitudinal position.

The method can further include providing a peel away sheath (PAS) that is coupled to the DBS lead. The PAS can have an elongate orientation marker that extends longitudinally over at least a major portion of an intrabody length of the directional medical device. The method can further include aligning the elongate orientation marker of the PAS with the passive orientation marker on the DBS lead during the placement of the DBS lead to thereby facilitate identification of misalignment and/or twist from a desired orientation.

The identifying can be carried out to identify the orientation directly from the MRI image data using the trained model without requiring post-operative, post-implantation CT images and without using any template.

The mesh model can be a high-resolution mesh model that is shape-constrained and can be deformable based on geometry and topology of the directional medical device.

The mesh model can have a contiguous configuration of a plurality of triangular mesh elements. The intensity of artifact patterns can include intensity profiles perpendicular to the triangular mesh elements.

The method can include providing visual and/or audible output notifying when there is a deviation from a desired orientation of the DBS lead with respect to target tissue based on the determined orientation.

These and other embodiments will be described further below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a side perspective view of a distal end portion of a directional deep brain stimulation (DBS) lead with electrodes and a passive marker according to embodiments of the present invention.

FIG. 1B is an enlarged schematic view of the electrodes and passive marker in two-dimensions of those devices on the DBS lead of FIG. 1A.

FIG. 1C are different side views of the passive marker shown in FIG. 1A.

FIG. 1D is a schematic end view of the passive marker shown in FIG. 1C according to embodiments of the present invention.

FIG. 2A is a three-dimensional a high resolution (triangular) mesh representation of different segments of the DBS lead and components thereof according to embodiments of the present invention.

FIG. 2B is a three-dimensional deformable mesh representation of the DBS lead and components thereof according to embodiments of the present invention.

FIG. 3 is a schematic illustration of an example mesh configuration for the mesh representation according to embodiments of the present invention.

FIG. 4 is a schematic illustration of a portion of a DBS lead illustrating that the passive marker of a directional DBS lead with reproducible CT artifacts for identifying lead orientation (Anterior, Posterior, Right, Left) as illustrated by the arrow in the top circle.

FIG. 5A is an MRI slice of an implanted lead in a brain of a subject.

FIG. 5B is a CT slice at a corresponding location of the MRI slice shown in FIG. 5A.

FIG. 5C is an overlay of the MRI and CT slices of FIGS. 5A and 5B.

FIG. 6A is another MRI slice of the implanted lead.

FIG. 6B is a CT slice at a corresponding location of the MRI slice shown in FIG. 6A.

FIG. 6C is an overlay of the MRI and CT slices of FIGS. 6A and 6B.

FIG. 7 is a three-dimensional MRI image fused with a post-operative CT image with a shape-constrained deformable brain model according to embodiments of the present invention.

FIGS. 8A-8C illustrate fused MRI and CT images with a shape-constrained deformable brain model used to identify the boundary of the thalamus where the DBS lead(s) are implanted whereby orientation of the leads can be extracted from the CT artifact created by the passive orientation marker according to embodiments of the present invention.

FIG. 9 is a side perspective view of a peel-away sheath releasably coupled to the directional DBS lead with an elongate marker according to embodiments of the present invention.

FIG. 10 is an enlarged partial side perspective view of a DBS lead with passive markers including an elongate passive marker that can be aligned with the elongate marker of the peel-away sheath shown in FIG. 9 according to embodiments of the present invention.

FIG. 11A is a top perspective view of a portion of a trajectory guide assembly comprising one or more channels for use in inserting the DBS lead into the brain for a suitable trajectory path according to embodiments of the present invention.

FIGS. 11B-11E are side perspective views of example trajectory guide assemblies incorporating the portion of the trajectory guide assembly shown in FIG. 11A according to embodiments of the present invention.

FIG. 12 is a flow chart of example actions that can be used to identify lead position and/or orientation according to embodiments of the present invention.

FIG. 13 is an example fused MRI/CT image data and registered mesh representation of a lead with a trajectory according to embodiments of the present invention.

FIG. 14 is another flow chart of example actions using anatomy based-orientation that can be used to identify a desired lead position and/or orientation according to embodiments of the present invention.

FIG. 15A is an example shape-constrained brain model applied to segment anatomical structures in an MRI scan according to embodiments of the present invention.

FIG. 15B is an example image of a sub-set of anatomical structures of interest relevant to a target treatment site in the brain according to embodiments of the present invention.

FIG. 15C is an example rendered image of a mesh representation of a model of the lead placed using quantitative indices to define optimal lead position and orientation according to embodiments of the present invention.

FIG. 16 is a schematic illustration of an image-guided surgical system according to some embodiments of the present invention.

FIG. 17 is a schematic illustration of an example data processing system according to some embodiments of the present invention.

### DETAILED DESCRIPTION

The present invention will now be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. The abbreviation “FIG.” may be used interchangeably with “Fig.” and the word “Figure” in the specification and figures. It will be appreciated that although discussed with respect to a certain embodiment, features or operation of one embodiment can apply to others.

In the drawings, the thickness of lines, layers, features, components and/or regions may be exaggerated for clarity and broken lines (such as those shown in circuit of flow diagrams) illustrate optional features or operations, unless specified otherwise. In addition, the sequence of operations (or steps) is not limited to the order presented in the claims unless specifically indicated otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when a feature, such as a layer, region or substrate, is referred to as being “on” another feature or element, it can be directly on the other element or intervening elements may also be present. In contrast, when an element is referred to as being “directly on” another feature or element, there are no intervening elements present. It will also be understood that, when a feature or element is referred to as being “connected” or “coupled” to another feature or element, it can be directly connected to the other element or intervening elements may be present. In contrast, when a feature or element is referred to as being “directly connected” or “directly coupled” to another element, there are no intervening elements present. Although described or shown with respect to one embodiment, the features so described or shown can apply to other embodiments.

The term “computer system” refers to any computer system and can include one or more processors, databases and servers. The computer system can comprise a local area network (LAN), a wide area network (WAN) and/or the internet. The computer system can comprise and/or be provided as a cloud computing resource. The computer system can comprise software and hardware and can reside at least partially on a workstation of a surgical planning and/or image-guided surgical system.

The term “brain atlas” refers to a digital model of features of a brain, human brain for human uses and animal brains for respective animal uses. The image-guided surgical systems contemplated by the present invention can be independent of any particular atlas. One example brain atlas is the WayPoint™ Navigator Software (manufacturer: FHC) which has an integrated brain atlas to assist with surgical planning and predictive modelling for DBS, LITT and epilepsy procedures. See https://www.fh-co.com/product/waypoint-navigator-software. Another example is the NeuroQuant® Software (manufacturer: Cortech Labs) which has an integrated brain atlas to automatically detect 3D anatomical structures from MR scans for purposes of planning and neurological assessment. See, https://www.cortechslabs.com/products/neuroquant/. The content of the noted websites are hereby incorporated by reference as if recited in full herein. The brain atlas can be linked or referenced rather than included in an onboard library of the surgical system.

The term “ACPC coordinate space” refers to a right-handed coordinate system defined by anterior and posterior commissures (AC, PC) and Mid-Sagittal plane points, with positive directions corresponding to a patient's anatomical Right, Anterior and Head directions with origin at the mid-commissure point.

The term “grid” refers to a pattern of crossed lines or shapes used as a reference for locating points or small spaces, e.g., a series of rows and intersecting columns, such as horizontal rows and vertical columns (but orientations other than vertical and horizontal can also be used). The grid can include associated visual indicia such as alphabetical markings (e.g., A-Z and the like) for rows and numbers for columns (e.g., 1-10) or the reverse. Other marking indicia may also be used. The grid can be provided as a flexible patch that can be releasably attached to the skull or scalp of a patient. For additional description of suitable grid devices, see co-pending, co-assigned U.S. patent application Ser. No. 12/236,621.

The term “fiducial marker” refers to a marker that can be electronically identified using image recognition and/or electronic interrogation of image data. The fiducial marker can be provided in any suitable manner, such as, but not limited to, a geometric shape of a portion of the tool, a component on or in the tool, a coating or fluid-filled component or feature (or combinations of different types of fiducial markers) that, for MRI/CT uses, makes the fiducial marker(s) visible in a respective imaging modality with sufficient signal intensity (brightness) for identifying location and/or orientation information for the tool and/or components thereof in space.

The terms “RF safe” and “MRI compatible” means that the so-called component(s) is safe for use in an MRI environment and as such is typically made of a non-ferromagnetic MRI compatible material(s) suitable to reside and/or operate in a high magnetic field environment, without inducing unplanned current that inadvertently unduly heats local tissue or otherwise interferes with the planned therapy.

The term “high-magnetic field” refers to field strengths above about 0.5 T, typically above 1.0T, and more typically between about 1.5T and 10T, including 3T systems. MRI Scanners are well known and include high-field closed bore and open bore systems.

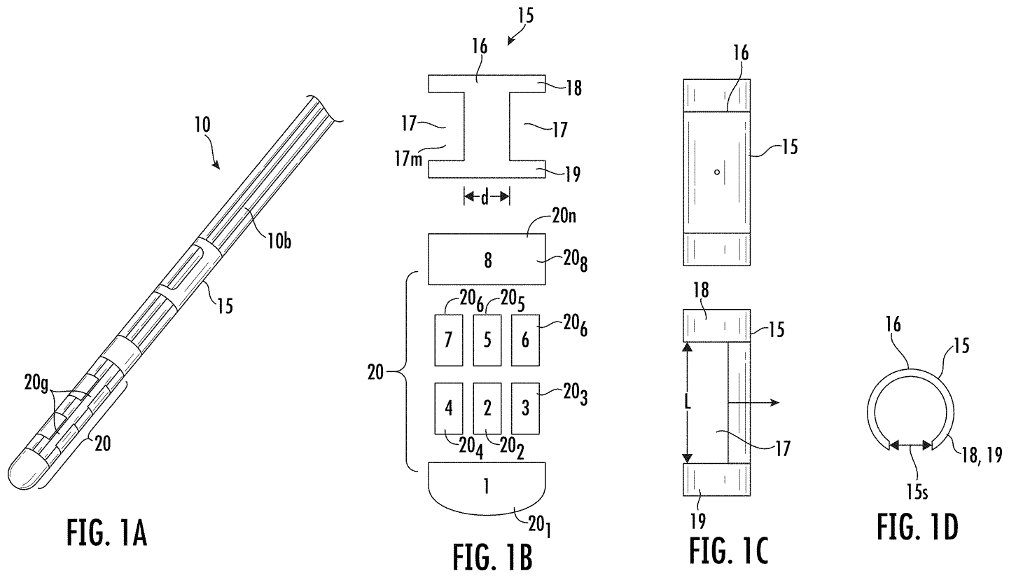

FIG. 1A illustrates a directional medical device 10 with a body 10b and at least one passive (orientation) marker 15. The term “directional” refers to a medical device that has a desired circumferential (rotational) orientation and/or longitudinal position with respect to target anatomy. The passive marker 15 is an orientation marker that creates a specific intensity pattern in image scans, such as CT image scans. In the example embodiment shown, the directional medical device 10 is a stimulation lead with electrode 20, particularly suitable for a deep brain stimulation lead. However, other directional medical devices 10 may be used, such as, for example, ablation catheters, drug delivery devices or tissue removal, e.g., biopsy or aspiration devices. The directional medical devices 10 may be for neurology targets or spinal or cardiac targets and may be particularly suitable for directional devices that are flexible or semi-rigid (able to bend or twist under a force of 1 inch-pound or more) or without a self-supporting longitudinal shape, typically with an outer diameter of 12F or less, so that they may torque or circumferentially or axially twist or otherwise displace during implantation.

FIG. 1B shows the electrode 20 and at least one passive marker 15 in a two-dimensional representation. FIG. 1C illustrates different side views (rotated 90 degrees from each other) of the passive marker 15 shown in FIGS. 1A and 1B. In some embodiments, the passive marker 15 includes a first longitudinally extending side portion 16 that defines a primary marker surface. The first side portion 16 can have a circumferential extent or distance “d” that is less than 180 degrees, typically 30-90 degrees, as shown in FIG. 1B. The first side portion 16 can merge into adjacent side portions 17 that have upper and lower portions 18, 19, respectively, that bound a medial portion 17m that is devoid of a marker surface as shown in FIGS. 1B and 1C. In some embodiments, the medial portion 17m can have a length “L” that is greater than a length of the top and bottom portions 18, 19. As shown in FIG. 1D, the passive marker 15 can have a longitudinally extending gap space 15s that resides along an entire length of the marker 15 between opposing laterally spaced apart ends 18, 19.

In the embodiment shown, the electrode 20 is configured to have a plurality of discrete electrode contact areas 20 at least some of which are longitudinally spaced apart and circumferentially spaced apart from at least some others. As shown, there are eight electrode areas (defining discrete tissue contact points) with a first set of electrode areas 205, 206, 207 provided at a first longitudinal position and circumferentially spaced apart from each other and a second set of electrode areas 202, 203, 204 provided at a second longitudinal position, spaced distally further from the first set of electrode areas, to reside closer to a most distal electrode area 201. The second set of electrode areas 202, 203, 204 can also be circumferentially spaced apart from each other.

The contact areas are conductive, such as metal plates, that are used to drive current through target tissue, such as brain tissue. For example, in FIG. 1B, 20₂, 20₃, and 20₄ are three contact areas (points) oriented at 120 degrees. That spacing allows for directional stimulation. An electrode 20 can have different configuration of contact points depending on the manufacturer and intended purpose. Also, more than one electrode 20 may be provided.

The electrode 20 can also include a most proximal electrode area 20₈ that can have a circumferential extent that is the same or greater than the first set of electrode areas or the second set of electrode areas. As shown, the proximal electrode area (contact area) 20₈ is a contact area/point implemented as a full ring that stimulates in 360 degrees. While the example medical device 10 shows the electrode 20 with eight electrode area contact points, other contact areas configurations can be provided and the contact areas can be provided in different numbers 20n, where n is the number of electrode contact areas, and is typically greater than or equal to 1 and less than 100, more typically between 1-10, 2-20, for example.

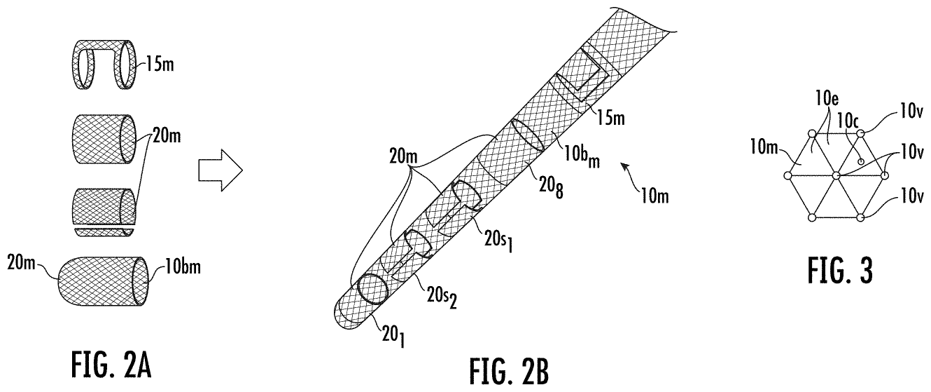

Referring to FIGS. 2A and 2B, a mesh representation 10m of at least a distal end portion of a directional medical device 10, such as a DBS lead with electrode contact areas 20n, can be provided. The mesh representation 10m can be created from a CAD model of the directional medical device 10 and can include mesh representations of the lead body 10bₘ, the electrodes 20m and passive marker 15m, integrated on a mesh representation 10m of the lead 10 as illustrated in FIGS. 2A, 2B.

FIG. 3 illustrates that the mesh representation 10m can comprise a set of mesh elements 10e configured with adjacent vertices 10v connected and with the mesh elements 10e shaped as triangular mesh elements, as shown. The mesh elements can have other geometric shapes. The surface mesh elements can comprise quadrilateral or simplex meshes or others polygon mesh elements. The mesh elements 10e can have centers of mass 10c and corners or vertices 10v.

The mesh representation 10m can define the geometry of the medical device 10, the geometry of the electrode contact points/areas and the medical device orientation.

The mesh representation 10m can be a high-resolution mesh representation. Each mesh element 10e can have a surface area that is less than a millimeter, such as a surface area in a range of 0.01 mm and 1 mm, for sub-millimeter resolution.

The mesh representation 10m is a deformable mesh representation so that respective mesh elements can elongate and/or compress within the body of the mesh representation to correspond to a position of a corresponding part of the medical device. The mesh representation 10m of the directional medical device 10 (e.g., lead) can be provided as a shape-constrained model of the directional medical device 10 (e.g., DBS lead). See, WEESE et al. “Shape-Constrained Deformable Models and Applications in Medical Imaging” Shape Analysis in Medical Image Analysis, pages 151-184, Lecture Notes in Computational Vision and Biomechanics, Vol 14. Springer (2014), for further discussion of shape-constrained models for segmentation, the content of which is hereby incorporated by reference as if recited in full herein. Quantitative indices derived from the geometry of the mesh representation in the image(s) can be used to define the orientation of a directional medical device 10 (e.g., lead). The quantitative indices can include, for example, coordinates of mesh vertices, centers of mass, moment invariants, principal curvature, surface derivatives, etc. For example, the triangles adapt to similar intensity values in different images. Their vertices are pulled to the same intensity values in different scans. That enforces point-based correspondence between mesh elements 10e, such as triangular vertices. For example, if one triangular vertex moves to a location/orientation or landmark, the same vertex will go to the same location/orientation or landmark (the same or very close 3-D location) in scans of different subjects.

The shape-constrained model can be trained on intensity profiles perpendicular to mesh elements 10e, such as mesh triangles, in fused MRI/CT data. The training process can be carried out as machine learning to determine or “learn” an orientation of a directional medical device 10 (e.g., lead) from image artifacts normal to mesh elements 10e, such as triangle mesh elements 10e, corresponding to the passive orientation marker 15.

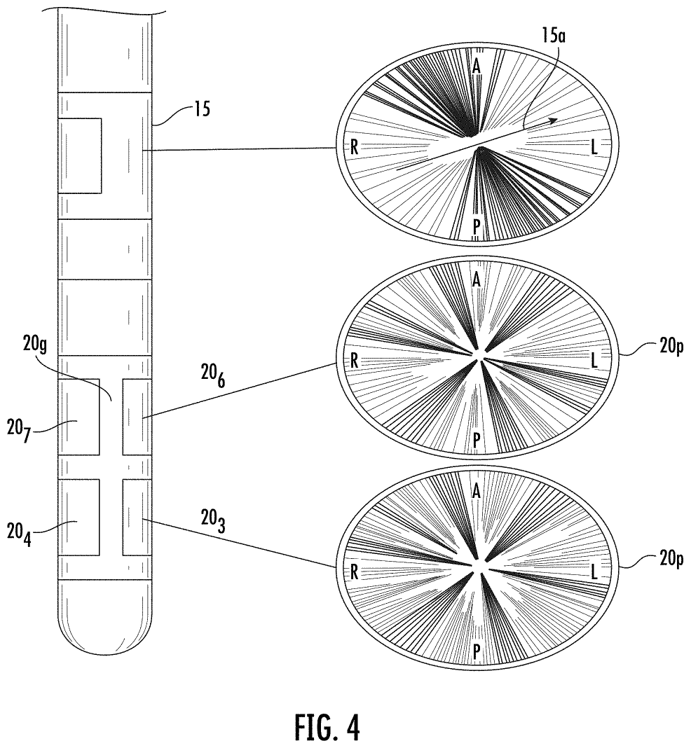

The passive orientation marker 15 creates a very specific intensity pattern 15p in CT scans as illustrated in FIGS. 4, 5A-5C and 6A-6C. As shown in FIG. 4, the passive marker 15 generates a CT image pattern 15p that defines anterior-posterior and right-left directions. These reproducible CT image artifacts can be detected reliably and used to identify lead orientation. See, Dembeck et al., Directional DBS leads show large deviations from their intended implantation orientation. Parkinsonism & related disorders, 67, pp. 117-121, 2019, the content of which is hereby incorporated by reference as if recited in full herein. The arrow 15a in the pattern 15p associated with the marker 15 in FIG. 4 is pointing between the anterior and the left directions. The patterns 20p of the electrodes 206, 207 and 203, 204 do not have the same orientation-distinctive data.

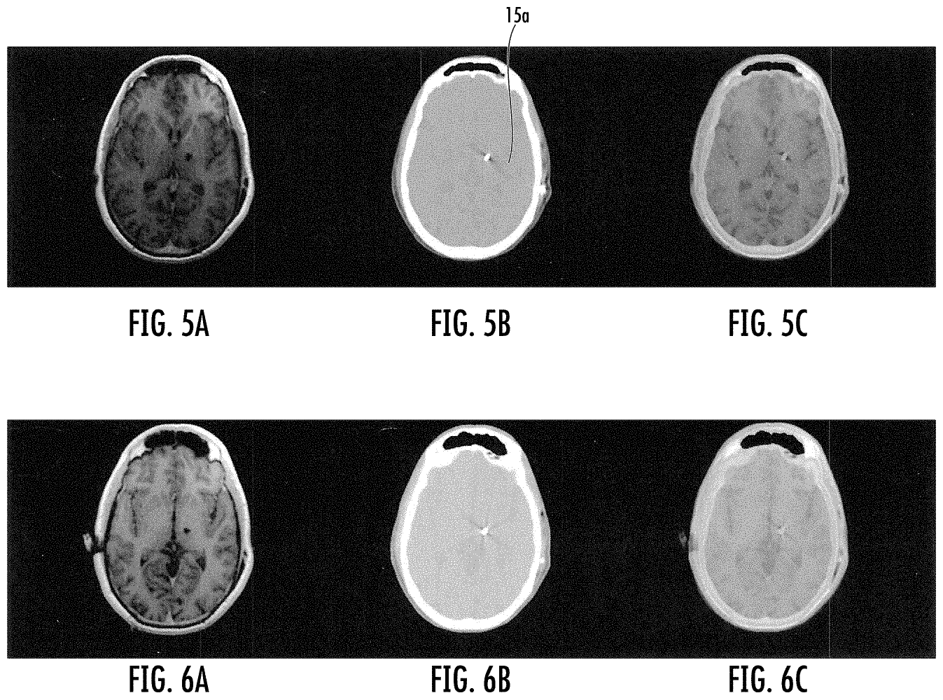

FIGS. 5A-5C and 6A-6C illustrate, from left to right, an MRI slice of an implanted DBS lead, a CT slice at the corresponding location, and an overlay of the MRI and CT slices. The unique CT image artifacts of the passive marker 15 (blue arrow, FIG. 5B) can be detected and used to identify the orientation of the implanted DBS lead, aligned to anatomical structure provided by the MRI slice. The pattern of the passive marker 15 in MRI image data can be different but directly correlated to orientation based on the CT image artifact pattern and the trained model. The correlations can be trained for different (future designs) of passive markers that create a distinct artifact pattern in the CT image that can be detected with image analysis/pattern recognition.

In some embodiments, once trained, the shape-constrained model of the directional medical device 10 (e.g., lead) can be adapted to be applied to intra- or post-operative MRI, to identify the orientation of the device 10 (e.g., lead) without the need for post-operative CT.

In some embodiments, the shape-constrained model can be trained only on CT data, and applied to post-operative CT image(s), to identify lead orientation for procedures performed outside of an MRI suite.

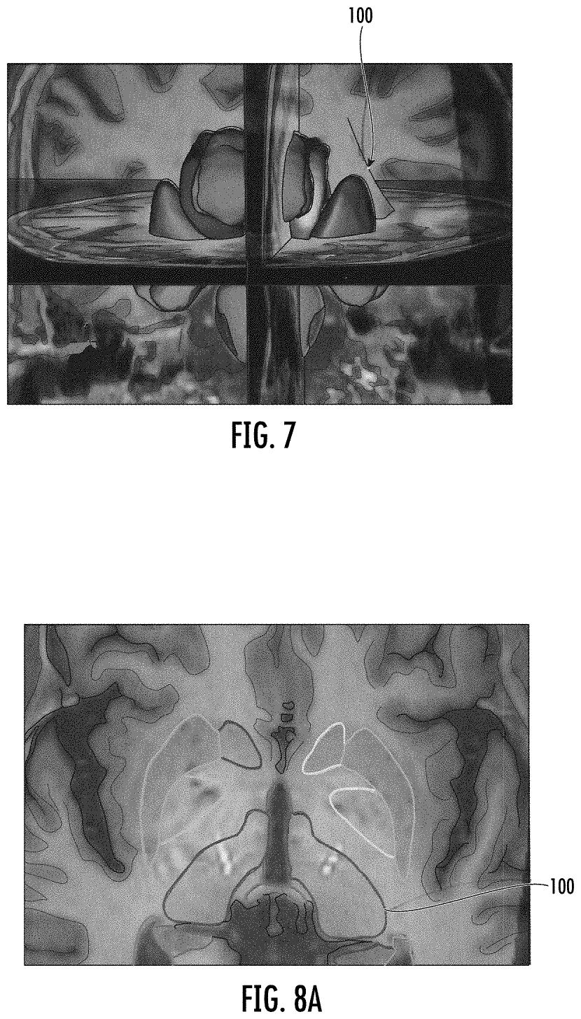

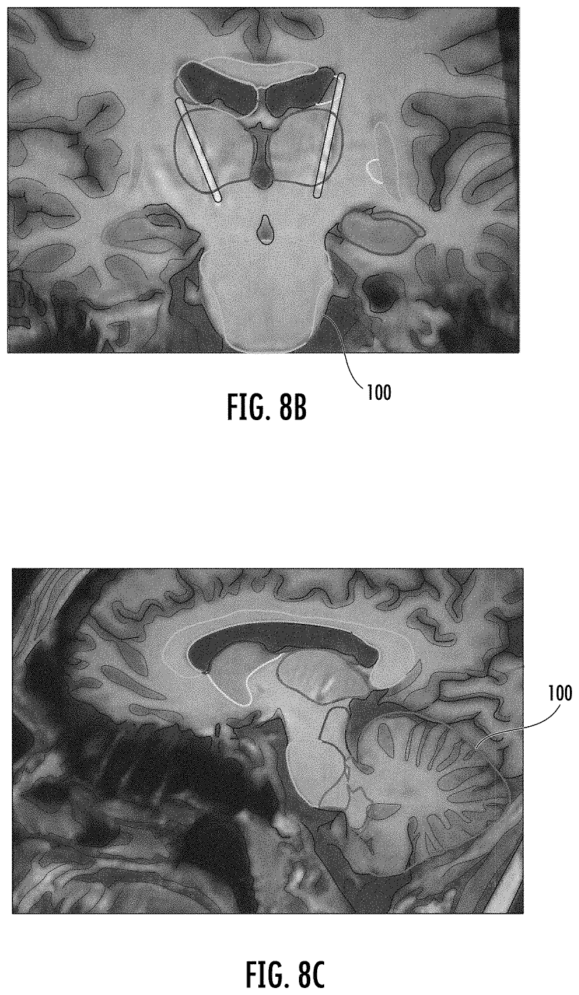

Referring to FIGS. 7 and 8A-8C, a preferred and/or desired orientation of the directional medical device 10 can be defined with respect to patient-specific anatomy using pre-operative MRI images. A shape-constrained, deformable (three-dimensional) brain model 100 can be adapted to the MRI data to extract patient-specific geometry of cortical and sub-cortical brain structures. An MRI fused with post-operative CT images and segmented with the shape-constrained, deformable brain model can be used to identify a boundary of the thalamus where the medical device 10, e.g., DBS lead(s) are implanted. The orientation of the medical device 10, e.g., leads, can be extracted from the CT artifact(s) pattern created by the passive orientation marker 15. Quantitative indices derived from the geometry of segmented brain structures can be used to define a desired orientation of a directional medical device 10 (e.g., lead). For example, this information can allow for programming operational output for target tissue, such as electrode pulse sequences of electrodes of DBS leads for target anatomical structure/tissue thereby providing optimal therapy. The quantitative indices can include, for example, coordinates of mesh vertices, centers of mass, moment invariants, principal curvature, surface derivatives, etc.

As discussed above with respect to the shape-constrained lead model, the shape-constrained protocol/methodology for the brain model can also provide unique point-based correspondence across different subjects unlike known methods. The mesh elements 10e, such as, for example, triangle shaped mesh elements, can adapt to similar intensity values in different images. Their vertices are pulled to the same intensity values in different scans. That enforces point-based correspondence between triangular vertices. For example, if one triangular vertex moves to an anatomical landmark, the same vertex will go to the same anatomical landmark (the same or very close 3-D location) in scans of different subjects.

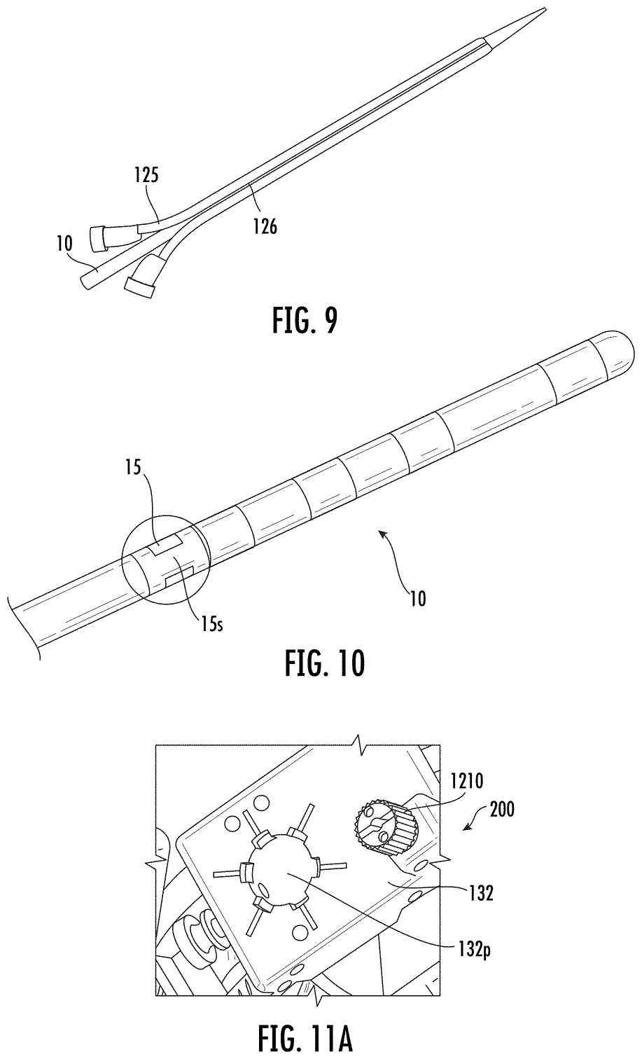

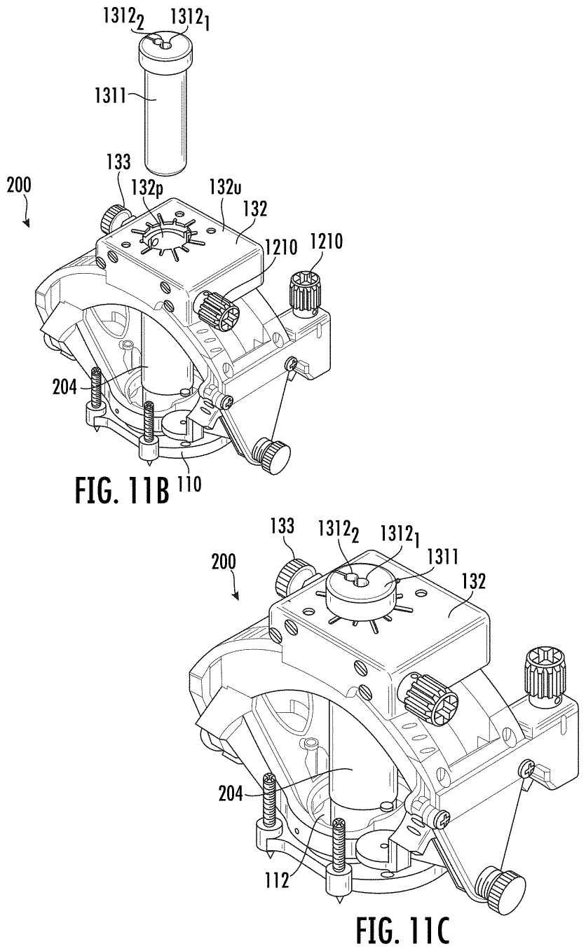

Referring to FIGS. 9 and 10, to guide a directional medical device 10 to a target with a specific orientation, a peel away sheath (PAS) 125 with an orientation marker 126 can be used. The orientation marker 126 can be a straight linear elongate marker that extends from a proximal end portion to a distal end portion, typically over an entire length or substantially the entire length (within +/−20%) of the sheath 125 about the directional medical device 10. The length of the orientation marker 126 can provide important information about twisting during implantation or closure and/or warning of an irregularity in case of undesired deviation, e.g., a rotation or twist of the marker 126 over a length of the sheath 125. The orientation marker 126 can comprise a radio opaque stripe that extends longitudinally. The passive orientation marker 15 of the medical device 10 can be aligned with the orientation marker 125 at a defined circumferential position, typically so that the orientation marker 126 extends between a longitudinally extending gap space 15s of the passive orientation marker 15 (FIG. 10) as the device 10 is inserted into the sheath 125 or via imaging before placement to ensure a specific orientation.

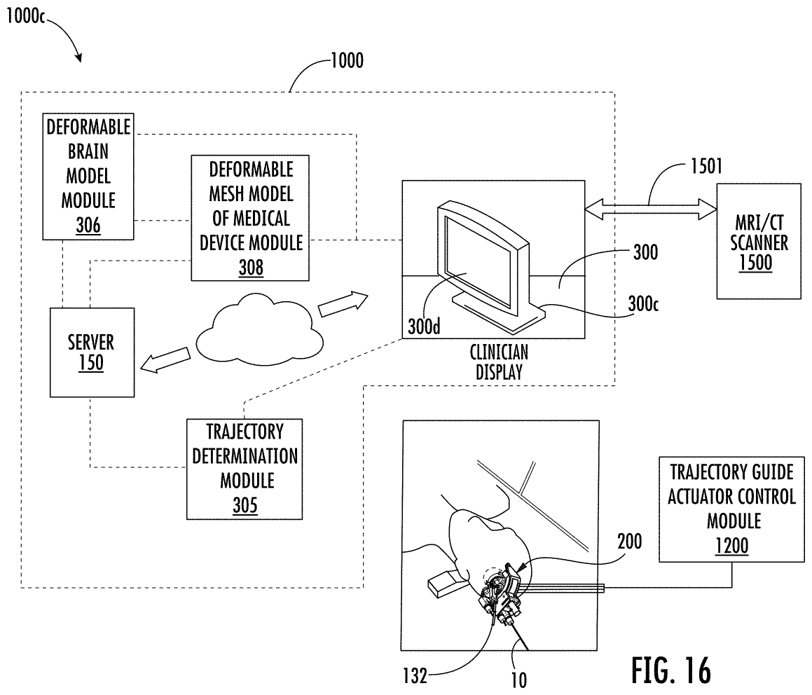

As will be discussed further below with respect to FIG. 16, embodiments of the present invention include an image-guided system 1000 with a trajectory guide 200 that can comprise at least one through channel that can be used to place the directional medical device 10. The system 1000 can use the deformable brain model 100 and/or directional medical device mesh representation model 10m to identify a suitable channel and/or channel orientation in a trajectory guide that can facilitate optimal placement.

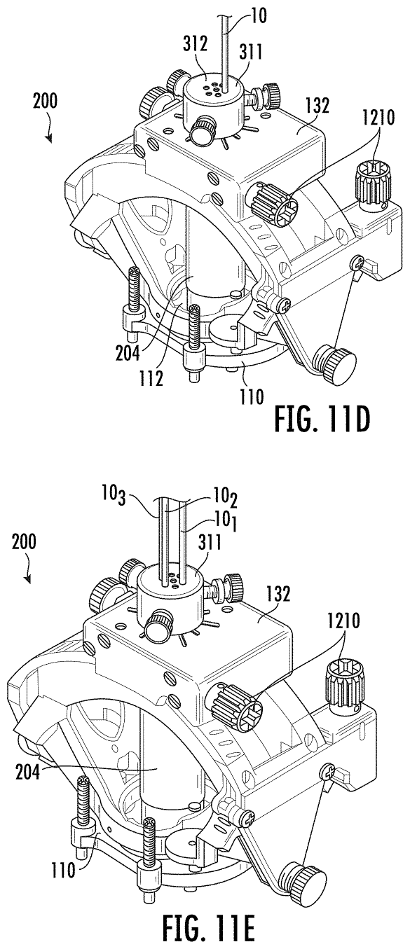

Referring to FIGS. 11A-11E, an example trajectory guide 200 is shown. The trajectory guide 200 can have a base 110 that surrounds a patient access aperture 112. The trajectory guide 200 can have a platform 132 with a port 132p. A tubular member 204 can extend below the port 132p and terminate adjacent to and/or above the patient access aperture 112. Orientation indicia 132i can be provided on the platform 132 that can be painted, coated or otherwise provided with color-coded markings on an upper surface 132u of the platform 132 that can help a user to align the guide 211 and/or identify a channel and/or path selection. FIGS. 11D-11E illustrate that a multi-lumen guide 311 with a plurality of spaced apart open through lumens/channels 312 can be coupled to the trajectory frame 200. FIGS. 11B and 11C illustrate the use of a rotatable device guide 1311 with at least one through channel 1312, shown as two 1312₁, 1312₂, one of which 1312₂ is offset from a centerline of the device guide. Rotating the device guide 1311 relative to the platform 132 can position the lumen/channel 1312₂ at different circumferential locations. The guides 311, 1311 can be used to place/insert medical devices 10 into the patient to the target site. In some embodiments, a plurality of medical devices 10₁, 10₂, 10₃ can be concurrently inserted through the trajectory frame 200 and into a patient/subject (FIG. 11E). The multi-lumen guide 311 can have the same number of channels 312 as a removable fluid filled guide and these channels 312 can be in the same position. As shown, there are seven channels 312 in the multiple lumen device guide 311. For more details of example multiple and single lumen device guides and trajectory guide assemblies, see, U.S. Pat. No. 10,905,498, the content of which is hereby incorporated by reference as if recited in full herein.

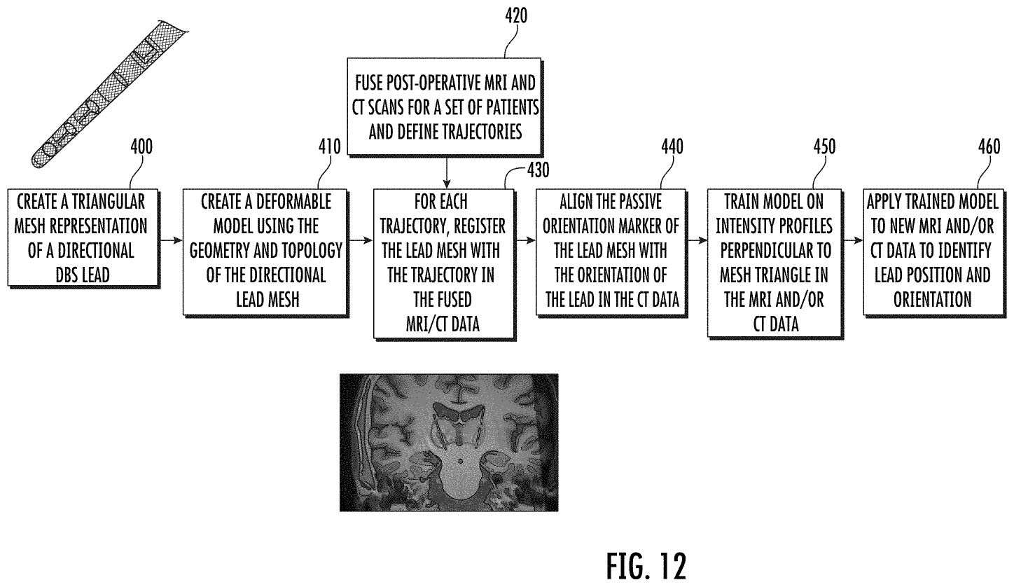

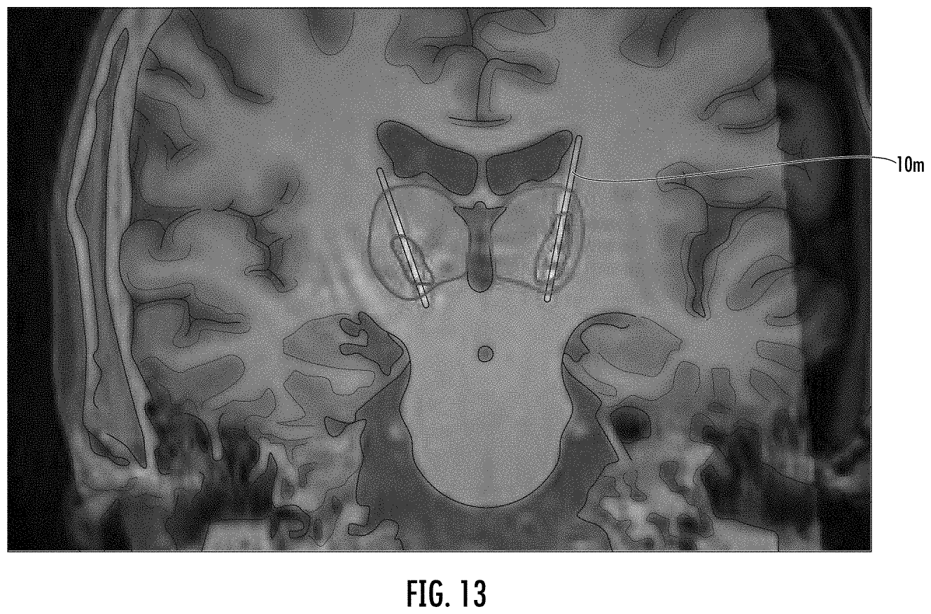

Referring to FIG. 12, an example set of actions are shown that can be used to non-invasively identify position and/or orientation of a medical device in the body. A triangular mesh representation of a directional medical device such as, for example, a DBS lead, is provided and/or created. A deformable model of the medical device, e.g., the DBS lead, is generated using the geometry and topology of the mesh representation of the directional lead (block 410). Post-operative MRI and CT scans for a set of patients are fused and used to define trajectories (block 420). For each trajectory, register the mesh representation of the lead with the trajectory in the fused MRI/CT data (block 430), FIG. 13. The passive orientation marker of the mesh lead is aligned with the orientation of the lead in the CT image data (block 440). A lead position and orientation model is trained based on intensity profiles perpendicular to mesh elements in the MRI and/or CT image data (block 450). The trained model is applied to new MRI and/or CT image data to identify lead position and orientation (block 460).

As discussed above, optionally, a peel-away sheath 125 with a radio opaque orientation marker 125, such as a stripe, can be aligned with the passive orientation marker 15 of a directional medical device 10, optionally a DBS lead, before or during implantation.

The actions/steps may be particularly suitable for identifying orientation of directional stimulation leads using a shape-constrained lead model trained on fused MRI/CT data. Further, unlike other methods (see, e.g., U.S. Pat Nos. 10,265,531, and 9,050,470, the contents of which are hereby incorporated by reference), no templates are used and in vivo orientation can be derived from the trained model and MRI image data directly, without the requirement to use and/or fuse post-operative CT image data to the MRI image data. Unlike the prior art, which requires a CT image to detect orientation. Embodiments of the present invention employ fused MRI/CT data to train a model. After the model is trained, the trained model can extract the lead orientation from MRI data alone. There is no need for a CT scan which reduces radiation exposure of the patient.

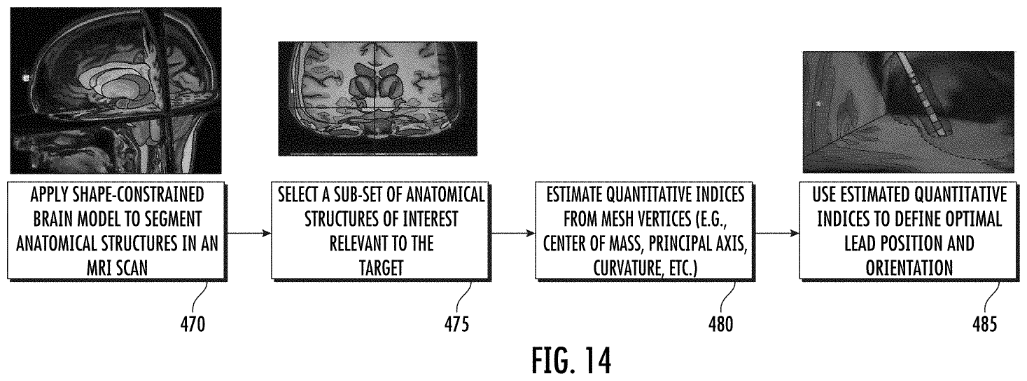

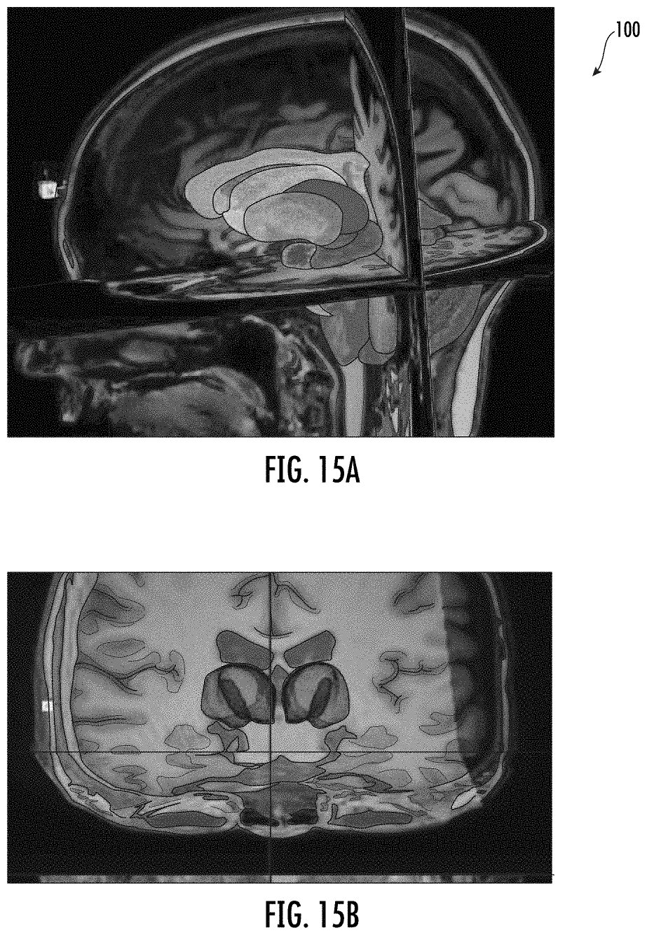

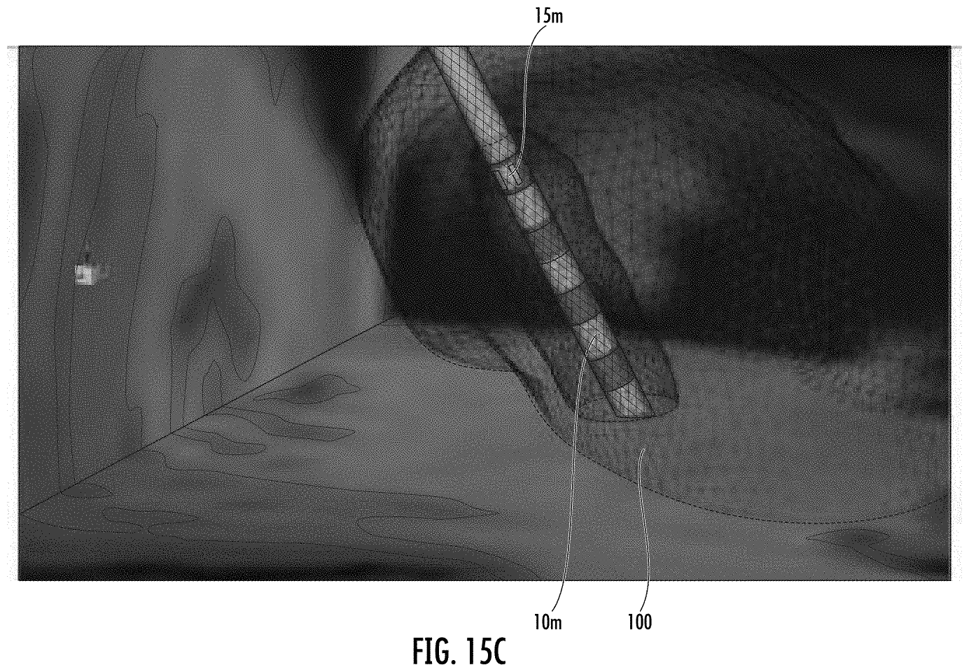

Referring to FIG. 14, embodiments of the present invention are directed to systems and methods for defining directional-device (e.g., DBS lead) orientation with respect to patient-specific anatomy (e.g., brain structures, fiber tracts, areas of functional activity, etc.). A shape-constrained brain model is applied to segment anatomical structures in an MRI scan (block 470), FIG. 15A. A sub-set of anatomical structures of interest in the brain model and relevant to the target are selected (block 475), FIG. 15B. Quantitative indices from mesh vertices (e.g., center of mass, principal axis, curvature, etc.) are estimated (block 480). The estimated quantitative indices are used to define a desired (e.g., optimal) medical device (e.g., lead) trajectory and/or position and orientation (block 485). A mesh representation 10m of the medical device (e.g., lead) can be rendered in the desired position and orientation in an image of the brain with the brain model of a patient for use in placing a corresponding physical lead, FIG. 15C.

The shape-constrained lead and/or the shape constrained brain model can be trained directly on post-operative CT data. Potentially, such data can be acquired with a portable CT scanner (O-arm), or a rotational X-ray system, and fused with an MRI during an implantation procedure. The X-ray/CT image can be obtained outside of the Operating Room (OR).

FIG. 16 illustrates an image-guided surgical system 1000. The surgical system 1000 comprises a computer system 1000c and can comprise or communicate with a clinician workstation 300 and at least one display 300d. The computer system 1000c can comprise at least one data processing circuit 300c.

The surgical system 1000 can include a trajectory determination module 305. The workstation 300 can communicate with a scanner 1500, such as an MRI and/or CT scanner via an interface 1501 that may be used to allow communication between the workstation 300 and the scanner 1500. The interface 1501 and/or circuit 300c may comprise hardware, software or a combination of same. The interface 1501 and/or circuit 300c may reside partially or totally in the scanner 1500, partially or totally in the workstation 300, or partially or totally in a discrete device(s) therebetween.

The workstation 300 and/or circuit 300c can passively or actively communicate with the scanner 1500. The system 1000 can also be configured to use functional patient data (e.g., fiber tracks, fMRI and the like) to help plan or refine a target surgical site. See, e.g., U.S. Pat. No. 8,315,689 for additional information on example workflows and surgical systems, the contents of which are hereby incorporated by reference as if recited in full herein.

Still referring to FIG. 16, the surgical system 1000 can comprise a server 150 as part of the computer system 1000c that can provide and/or be in communication with one or more modules such as, a trajectory determination module 305, a deformable brain model module 306, a deformable mesh model of the directional lead module 308 and or a trajectory guide actuator control module 1200. The workstation 300 can communicate with the server 150 via a computer network, such as one or more of local area networks (LAN), wide area networks (WAN) and can include a private intranet and/or the public internet (also known as the World Wide Web or “the web” or “the internet”). The server 150 can include and/or be in communication with one or more of the modules 306, 308, 305, 1200 using appropriate firewalls for HIPPA or other regulatory compliance.

The computer system 1000c and/or server 150 can be provided using cloud computing which includes the provision of computational resources on demand via a computer network. The resources can be embodied as various infrastructure services (e.g., compute, storage, etc.) as well as applications, databases, file services, email, etc. In the traditional model of computing, both data and software are typically fully contained on the user's computer; in cloud computing, the user's computer may contain little software or data (perhaps an operating system and/or web browser) and may serve as little more than a display terminal for processes occurring on a network of external computers. A cloud computing service (or an aggregation of multiple cloud resources) may be generally referred to as the “Cloud”. Cloud storage may include a model of networked computer data storage where data is stored on multiple virtual servers, rather than being hosted on one or more dedicated servers.

The image-guided system 1000 can be configured to carry out diagnostic and interventional procedures such as to guide and/or place interventional devices to any desired internal region of the body or object but may be particularly suitable for neurosurgeries. The object can be any object and may be particularly suitable for animal and/or human subjects. For example, the system can be used for gene and/or stem-cell based therapy delivery or other neural therapy delivery and allow user-defined custom targets in the brain or to other locations. In addition, embodiments of the systems can be used to ablate tissue in the brain or other locations and/or place electrode stimulation leads. In some embodiments, it is contemplated that the systems can be configured to treat AFIB in cardiac tissue, and/or to deliver stem cells or other cardio-rebuilding cells or products into cardiac tissue, such as a heart wall, via a minimally invasive MRI guided procedure while the heart is beating (i.e., not requiring a non-beating heart with the patient on a heart-lung machine).

Examples of known treatments and/or target body regions are described in U.S. Pat. Nos. 6,708,064; 6,438,423; 6,356,786; 6,526,318; 6,405,079; 6,167,311; 6,539,263; 6,609,030 and 6,050,992, the contents of which are hereby incorporated by reference as if recited in full herein.

The trajectory guide actuator control module 1200 can be used to automatically move actuators 1210 (FIGS. 11A-11E) of the trajectory guide 200 or provide movement directions for a user to manually move the actuators 1210 desired amounts to provide a selected candidate trajectory identified by the deformable brain model module 306 and/or trajectory determination module 306. The trajectory guide actuator control module 1200 can comprise MRI-compatible stepper motors that reside in a housing in an MRI scanner room, optionally coupled to the patient bed, so as to be able to move in and out of the bore of the magnet while coupled to the bed. See, e.g., U.S. Provisional Patent Application Ser. No. 62/988,609, filed Mar. 12, 2020, and U.S patent application Ser. No. 17/185,060, the contents of which are hereby incorporated by reference as if recited in full herein.

To be clear, embodiments of the invention can be provided as a separate data processing system and/or module(s) or combined data processing systems and/or modules. The data processing system can be compatible with MRI and/or CT systems.

The MRI scanner 1500 can include a console that has a “launch” application or portal for allowing communication to the circuit 300c of the workstation 300. The scanner console can acquire volumetric image data of a respective patient, such as, for example, T1-weighted (post-contrast scan) data or other image data (e.g., high resolution image data for a specific volume) of a patient's head and/or brain (or other target anatomy).

In some embodiments, the console can push DICOM images or other suitable image data to the workstation 300 and/or circuit 300c. The workstation 300 and/or circuit 300c can be configured to passively wait for data to be sent from the MR scanner 1500 and the circuit 300c/workstation 300 does not query the scanner or initiate a communication to the scanner. In other embodiments, a dynamic or active communication protocol between the circuit 300c/workstation 300 and the scanner 1500 may be used to acquire image data and initiate or request particular scans and/or scan volumes. Also, in some embodiments, pre-DICOM, but reconstructed image data, can be sent to the circuit 300c/workstation 300 for processing or display. In other embodiments, pre-reconstruction image data (e.g., substantially “raw” image data) can be sent to the circuit 300c/workstation 300 for Fourier Transform and reconstruction.

Embodiments of the invention are particularly useful for neurosurgeries such as deep brain surgeries. An end user, such as a neurosurgeon, can prepare, review and finalize inputs using a defined workflow. Once the inputs are provided (and also typically prepared and reviewed by a user), a defined set of rules can automatically determine orientation of a directional medical device to confirm no twisting or deviation and that data can be presented to the user, typically via a display of a computer system such as a clinician workstation.

The system 1000 can be configured to register CT image data and MRI image data by initiating automatic fusion of scans and allow/prompt a user to visually review image fusion results. Fusion tools can be used to allow a user to manually correct each incorrect fusion result. Automatic registration of a digital brain atlas to an MRI scan, such as a T1W scan, can be initiated and a user can be allowed/prompted to visually review registration results.

For identifying the potential entry points, all no-go regions in the brain (at least those within any proximity to entry sites and target treatment volumes) can be segmented or otherwise identified. The outer surface of the head can be virtually divided into defined sub-areas, optionally with maximal outer perimeter sides in a range of 0.1 mm-2 mm, such as about 1 mm square sub-areas. Entry can be defined by a grid applied to the head of a subject. See, co-pending U.S. Provisional Patent Application Ser. No. 63/018,215, filed Apr. 30, 2020, for a discussion of an automated trajectory planning system, the contents of which are hereby incorporated by reference as if recited in full herein.

Embodiments of the present invention may take the form of an entirely software embodiment or an embodiment combining software and hardware aspects, all generally referred to herein as a “circuit” or “module.” Furthermore, the present invention may take the form of a computer program product on a (non-transient) computer-usable storage medium having computer-usable program code embodied in the medium. Any suitable computer readable medium may be utilized including hard disks, CD-ROMs, optical storage devices, a transmission media such as those supporting the Internet or an intranet, or magnetic storage devices. Some circuits, modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller. Embodiments of the present invention are not limited to a particular programming language.

Computer program code for carrying out operations of data processing systems, method steps or actions, modules or circuits (or portions thereof) discussed herein may be written in a high-level programming language, such as Python, Java, AJAX (Asynchronous JavaScript), C, and/or C++, for development convenience. In addition, computer program code for carrying out operations of exemplary embodiments may also be written in other programming languages, such as, but not limited to, interpreted languages. Some modules or routines may be written in assembly language or even micro-code to enhance performance and/or memory usage. However, embodiments are not limited to a particular programming language. As noted above, the functionality of any or all of the program modules may also be implemented using discrete hardware components, one or more application specific integrated circuits (ASICs), or a programmed digital signal processor or microcontroller. The program code may execute entirely on one (e.g., a workstation) computer, partly on one computer, as a stand-alone software package, partly on the workstation's computer and partly on another computer, local and/or remote or entirely on the other local or remote computer. In the latter scenario, the other local or remote computer may be connected to the user's computer through a local area network (LAN) or a wide area network (WAN), or the connection may be made to an external computer (for example, through the Internet using an Internet Service Provider).

The present invention is described in part with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing some or all of the functions/acts specified in the flowchart and/or block diagram block or blocks.

The flowcharts and block diagrams of certain of the figures herein illustrate exemplary architecture, functionality, and operation of possible implementations of embodiments of the present invention. In this regard, each block in the flow charts or block diagrams represents a module, segment, or portion of code, which comprises one or more executable instructions for implementing the specified logical function(s). It should also be noted that in some alternative implementations, the functions noted in the blocks may occur out of the order noted in the figures. For example, two blocks shown in succession may in fact be executed substantially concurrently or the blocks may sometimes be executed in the reverse order or two or more blocks may be combined, depending upon the functionality. The AC, PC and MSP locations of images of a brain of respective patients can be identified in any suitable manner. For example, AC, PC and MSP locations can be identified through the digital brain atlas after it is registered with patient images.

Although not shown, in some embodiments, one or more of the directional medical devices 10 can be configured with one or more lumens and exit ports that deliver desired cellular, biological, and/or drug therapeutics to the target area, such as the brain. The tools may also incorporate transseptal needles, biopsy and/or injection needles as well as ablation devices. The lumens, where used, may receive extendable needles that may exit the probe from the distal end or from the sides, proximal, distal, or even, through the electrodes to precisely deliver cellular/biological therapeutics to the desired anatomy target. This delivery configuration may be a potential way to treat patients, where the cellular/biological therapeutics can be delivered into the desired anatomy to modify their cellular function. The cells (e.g., stem cells) may improve function. MRI can typically be effectively used to monitor the efficacy and/or delivery of the therapy.

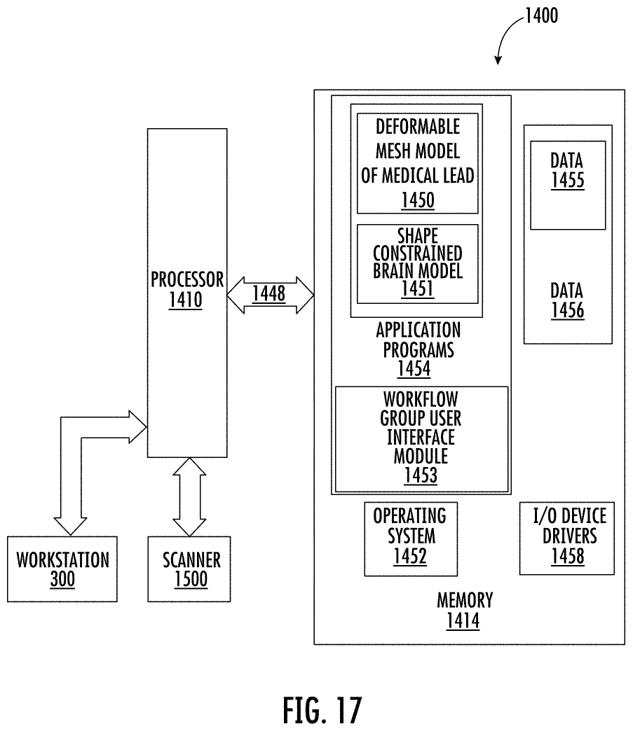

FIG. 17 is a schematic illustration of a data processing system 1400 that can be used with or define part of the image guided surgical system 1000. The data processing system may be incorporated in one or more digital signal processors in any suitable device or devices. As shown in FIG. 17, the processor 1410 can communicate with or be onboard the workstation 300 and/or can communicate with a scanner 1500 and with memory 1414 via an address/data bus 1448. The processor 1410 can be any commercially available or custom microprocessor. The memory 1414 is representative of the overall hierarchy of memory devices containing the software and data used to implement the functionality of the data processing system. The memory 1414 can include, but is not limited to, the following types of devices: cache, ROM, PROM, EPROM, EEPROM, flash memory, SRAM, and DRAM.

As shown in FIG. 17 the memory 1414 may include several categories of software and data used in the data processing system: the operating system 1452; the application programs 1454; the input/output (I/O) device drivers 1458; and data 1456. FIG. 17 also illustrates the application programs 1454 can include a Deformable mesh model of a medical device (e.g., lead) module 1450, a shape constrained brain model module 1451 and a workflow group User Interface Module 1453 (that facilitates user actions and provides user review of orientation and/or intrabody trajectories for example).

As will be appreciated by those of skill in the art, the operating systems 1452 may be any operating system suitable for use with a data processing system, such as OS/2, AIX, DOS, OS/390 or System390 from International Business Machines Corporation, Armonk, NY, Window versions from Microsoft Corporation, Redmond, WA, Unix or Linux or FreeBSD, Palm OS from Palm, Inc., Mac OS from Apple Computer, LabView, or proprietary operating systems. The I/O device drivers 1458 typically include software routines accessed through the operating system 1452 by the application programs 1454 to communicate with devices such as I/O data port(s), data storage 1456 and certain memory 1414 components. The application programs 1454 are illustrative of the programs that implement the various features of the data processing system and can include at least one application, which supports operations according to embodiments of the present invention. Finally, the data 1456 represents the static and dynamic data used by the application programs 1454, the operating system 1452, the I/O device drivers 1458, and other software programs that may reside in the memory 1414.

While the present invention is illustrated, for example, with reference to the Modules 1450, 1451 being application programs in FIG. 17, as will be appreciated by those of skill in the art, other configurations may also be utilized while still benefiting from the teachings of the present invention. For example, the Modules 1450, 1451 may also be incorporated into the operating system 1452, the I/O device drivers 1458 or other such logical division of the data processing system. Thus, the present invention should not be construed as limited to the configuration of FIG. 17 which is intended to encompass any configuration capable of carrying out the operations described herein. Further, one or more of modules, i.e., Modules 1450, 1451 can communicate with or be incorporated totally or partially in other components, such as a workstation, a scanner 1500 such as an MRI scanner, an interface device. Typically, the workstation 300 will include the modules 1450, 1451 and the scanner can include a module that communicates with the workstation 300 and can push image data thereto.

The Modules 1450, 1451 can be configured to carry out the methods of FIGS. 12 and/or 14, respectively. The Modules 1450, 1451 can correspond to modules 308, 306, respectively, in FIG. 16.

The I/O data port can be used to transfer information between the data processing system 1400, the computer system 1000c, the circuit 300c or workstation 300, the scanner 1500, and another computer system or a network (e.g., the Internet) or to other devices controlled by or in communication with the processor. These components may be conventional components such as those used in many conventional data processing systems, which may be configured in accordance with the present invention to operate as described herein.

In the drawings and specification, there have been disclosed embodiments of the invention and, although specific terms are employed, they are used in a generic and descriptive sense only and not for purposes of limitation, the scope of the invention being set forth in the following claims. Thus, the foregoing is illustrative of the present invention and is not to be construed as limiting thereof. More particularly, the workflow steps may be carried out in a different manner, in a different order and/or with other workflow steps or may omit some or replace some workflow steps with other steps. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. In the claims, means-plus-function clauses, where used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. An image-guided surgical system, comprising:
a workstation comprising a display;
a computer system in communication with or at least partially onboard the workstation, wherein the computer system is configured to:
provide a mesh model of a directional medical device; and
determine an intrabody orientation of the directional medical device from CT and/or MRI image data.

2. The system of claim 1, wherein the mesh model is a shape-constrained trained model of fused post-operative MRI and CT image data of a set of patients having implanted respective directional medical devices using intensity of artifact patterns corresponding to a passive orientation marker on the respective directional medical devices.

3. The system of claim 1, wherein the directional medical device is a deep brain stimulation lead with a plurality of circumferentially spaced apart electrodes aligned in at least one longitudinal position of the directional medical device.

4. The system of claim 1, wherein the system comprises the directional medical device and a peel away sheath (PAS) coupled to the directional medical device, and wherein the PAS comprises an elongate orientation marker that extends longitudinally over at least a major portion of an intrabody length of the directional medical device.

5. The system of claim 2, wherein the computer system is configured to determine the intrabody orientation directly from new MRI image data using the trained model without requiring post-operative, post-implantation CT images and without using any template.

6. The system of claim 2, wherein the mesh model is a high-resolution mesh model that is shape-constrained and deformable and is defined based on geometry and topology of the directional medical device.

7. The system of claim 6, wherein the mesh model comprises a contiguous configuration of a plurality of triangular mesh elements, and wherein the intensity of artifact patterns are intensity profiles perpendicular to the triangular mesh elements.

8. The system of claim 1, wherein the computer system is configured to generate output identifying a deviation from a desired orientation of the directional medical lead with respect to target tissue based on the determined orientation.

9. The system of claim 1, wherein the computer system is further configured to:
provide a mesh brain model comprising vertices;
select a subset of anatomical brain structures of interest relevant to a target intrabody site using the mesh brain model;
evaluate quantitative indices derived from at least some of the mesh vertices; and
define a patient-specific anatomy-based trajectory and/or a position and orientation of a directional medical device using the quantitative indices.

10. The system of claim 9, wherein the quantitative indices comprise one or more of: a center of mass, a principal axis and a curvature of the mesh brain model and/or mesh elements thereof

11. The system of claim 1, in combination with a CT or MRI scanner, wherein the workstation is in communication with the CT or MRI scanner, wherein the workstation comprises a DICOM interface that receives images from the CT or MRI scanner to provide the at least one image for the surgical system.

12. A method of identifying a deep brain stimulation lead orientation, comprising:
electronically providing a mesh brain model comprising vertices;
selecting a subset of anatomical brain structures of interest relevant to a target intrabody site using the mesh brain model;
evaluating quantitative indices derived from at least some of the mesh vertices; and
electronically defining a patient-specific anatomy-based trajectory and/or a position and orientation of the DBS lead using the quantitative indices.

13. The method of claim 12, wherein the quantitative indices comprise one or more of: a center of mass, a principal axis and a curvature of the mesh brain model and/or mesh elements thereof

14. A method of identifying lead orientation of a deep brain stimulation (DBS) lead, comprising:
providing a mesh model of the DBS lead, wherein the mesh model is a shape-constrained trained model of fused post-operative MRI and CT image data of brains from a set of patients defined by intensity of artifact patterns corresponding to a passive orientation marker on respective actual corresponding to the DBS leads;
placing a DBS lead in a brain of a patient using an MRI image-guided surgical system;
obtaining MRI image data of the brain of the patient;
applying the mesh model to the MRI image data; and
identifying orientation of the DBS lead in the brain of the patient using the applied mesh model.

15. The method of claim 14, wherein the DBS lead comprises a passive orientation marker longitudinally spaced apart from a plurality of circumferentially spaced apart electrodes, and wherein the circumferentially spaced apart electrodes are aligned in at least one longitudinal position.

16. The method of claim 15, further comprising providing a peel away sheath (PAS) that is coupled to the DBS lead, wherein the PAS comprises an elongate orientation marker that extends longitudinally over at least a major portion of an intrabody length of the directional medical device, and wherein the method further comprises aligning the elongate orientation marker of the PAS with the passive orientation marker on the DBS lead during the placement of the DBS lead to thereby facilitate identification of mis-alignment and/or twist from a desired orientation.

17. The method of claim 14, wherein the identifying is carried out to identify the orientation directly from the MRI image data using the trained model without requiring post-operative, post-implantation CT images and without using any template.

18. The method of claim 14, wherein the mesh model is a high-resolution mesh model that is shape-constrained and deformable based on geometry and topology of the directional medical device.

19. The method of claim 18, wherein the mesh model comprises a contiguous configuration of a plurality of mesh elements, and wherein the intensity of artifact patterns are intensity profiles perpendicular to the mesh elements, optionally wherein the mesh elements are triangular mesh elements.

20. The method of claim 14, further comprising providing visual and/or audible output notifying when there is a deviation from a desired orientation of the DBS lead with respect to target tissue based on the determined orientation.
